(19) United States
(12) Patent Application Publication
(10) Pub. No.: US 2026/0069868 A1
(43) Pub. Date: Mar. 12, 2026

(54) Systems and Methods for Wirelessly Powered Sources with Programmable Amplitude and Spectrum Spreading Using Pulse Width Modulation

(71) Applicant: The Regents of the University of California, Oakland, CA (US)

(72) Inventor: Aydin Babakhani, Los Angeles, CA (US)

(73) Assignee: The Regents of the University of California, Oakland, CA (US)

(21) Appl. No.: 19/109,537

(22) PCT Filed: Sep. 6, 2023
(86) PCT No.: PCT/US2023/073562

§ 371 Date: Mar. 6, 2025

Related U.S. Application Data
(60) Provisional application No. 63/374,843, filed on Sep. 7, 2022.
(60) Provisional application No. 63/378,336, filed on Oct. 4, 2022.

(51) Int. Cl. A61N 1/372; A61N 1/378

(52) U.S. Cl. CPC A61N 1/37211; A61N 1/3787

(57) ABSTRACT

Systems and methods of wirelessly powered stimulators and implantable pulse generators that can generate stimulations with programmable amplitudes and RF spectrum control in the RF domain are described. An embodiment includes an implantable pulse generator that includes: an Rx antenna configured to receive a radio frequency (RF) signal: a demodulator coupled to the Rx antenna, the rectifier, and a source, and configured to control an output of a train of mini-pulses, based on the RF signal, each mini-pulse having a voltage amplitude and a pulse width (on-portion) smaller than a pulse width TO; and stimulation circuitry 250 coupled to receive the train of minipulses and deliver the train of mini-pulse to an electrode, where the train of mini-pulses to the electrode effectively corresponds to a stimulation pulse, having a pulse width TO and an amplitude that is a function of the pulse widths of the mini-pulses.

### CROSS REFERENCE TO RELATED APPLICATIONS

The current application claims priority to U.S. Provisional Patent Application No. 63/374,843, entitled “Systems and Methods for Wirelessly Powered Sources with Programmable Amplitude and Spectrum Spreading Using Pulse Width Modulation”, filed Sep. 7, 2022, and U.S. Provisional Patent Application No. 63/378,336, entitled “Systems and Methods for Wirelessly Powered Sources with Programmable Amplitude and Spectrum Spreading Using Pulse Width Modulation” filed Oct. 4, 2022, the disclosures of which are hereby incorporated by reference herein in their entireties.

### FIELD OF THE INVENTION

The present invention generally relates to wirelessly powered stimulators and implantable pulse generators (IPG). In particular, to stimulators that can generate stimulations with programmable amplitudes.

### BACKGROUND OF THE INVENTION

Stimulators, such as implantable pulse generators (IPGs) have solved various critical clinical problems and improved the quality of human life. Their applications can include chronic pain relief, motor function recovery for spinal cord injuries, the treatment of gastroesophageal reflux disease, cardiac pacemaking, and curing stress urinary incontinence, among various other applications. Conventional IPGs are bulky with the battery taking up most of the unit, and the necessary leads are prone to cause various complications.

### SUMMARY OF THE INVENTION

Systems and methods of wirelessly powered stimulators and implantable pulse generators that can generate stimulations with programmable amplitudes in accordance with embodiments of the invention are described. An embodiment includes an implantable pulse generator that includes: an Rx antenna configured to receive a radio frequency (RF) signal; a rectifier coupled to the Rx antenna and configured to rectify the RF signal to generate an output voltage VDD; an energy storage capacitor coupled to the rectifier and configured to be charged by the output voltage VDD; a demodulator coupled to the Rx antenna, the rectifier, and a source, and configured to control an output of a train of mini-pulses, based on the RF signal, each mini-pulse having a voltage amplitude and a pulse width (on-portion) smaller than a pulse width TO; and stimulation circuitry 250 coupled to receive the train of mini-pulses and deliver the train of mini-pulse to an electrode, the stimulation circuitry configured such that delivery of the train of mini-pulses to the electrode effectively corresponds to a stimulation pulse, having the pulse width TO and an amplitude that is a function of the pulse widths of the mini-pulses included in the train of mini-pulses.

In a further embodiment, the RF signal is amplitude modulated to include several notches, and the demodulator is configured to detect the several notches and, for each notch, to enable the output of a mini-pulse in the train of mini-pulses.

In a further embodiment, the demodulator is configured to control a release of energy stored in an energy storage capacitor to output the train of mini-pulses.

In a further embodiment, the demodulator is configured to control the coupling of the output voltage VDD to output the train of mini-pulses.

In a further embodiment, the stimulation circuitry includes a low pass filter.

In a further embodiment, the low pass filter incldues a resistor 252 and a capacitor that are added.

In a further embodiment, the stimulation circuitry incldues a DC-block capacitor coupled to the output of the low pass filter, where the DC-block capacitor prevents release of DC charge.

In a further embodiment, each mini-pulse includes an on portion and an off portion, the on portion corresponding to the pulse width of the mini-pulse; and the pulse width TO of the stimulation pulse is greater than a summation of the on portions of the mini-pulses in the train of mini-pulses.

In a further embodiment, each mini-pulse includes an on portion and an off portion, the on portion corresponding to the pulse width of the mini-pulse; and the pulse width TO of the stimulation pulse is substantially equal to a summation of the on portion and the off portion of each mini-pulse in the train of mini-pulses.

In a further embodiment, each mini-pulse includes an on portion and an off portion, the on portion corresponding to the pulse width of the mini-pulse; and the amplitude of the stimulation pulse is proportional to a summation of the pulse widths of the mini-pulses in the train of mini-pulses.

In a further embodiment, the voltage amplitudes of the mini-pulses in the train of mini-pulses are constant.

In a further embodiment, the pulse widths of the mini-pulses in the train of mini-pulses are constant.

In a further embodiment, the pulse widths of the mini-pulses in the train of mini-pulses vary.

In a further embodiment, the pulse widths of the mini-pulses in the train of mini-pulses alternate between a first pulse width and a second pulse width.

In a further embodiment, a first subset of the mini-pulses in the train of mini-pulses have a first pulse width and a second subset of the mini-pulses in the train of mini-pulses have a second pulse width different from the first pulse width.

In a further embodiment, the train of mini-pulses is configurable to provide different spectrums in the frequency domain, wherein a spectrum of a train of mini-pulses in the frequency domain changes based on a configuration of the train of mini-pulses.

In a further embodiment, wherein a spectrum of the RF signal changes in the frequency domain, wherein the stimulation circuitry generates a same stimulation pulse having the pulse width TO.

In a further embodiment, the stimulation circuitry is coupled to receive different trains of mini-pulses in the frequency domain and generate a same stimulation pulse having the pulse width TO and an amplitude that is a function of the pulse widths of the mini-pulses included in the different trains of mini-pulses.

Another embodiment includes a method of delivering a stimulation pulse having a pulse width TO and an amplitude, that includes: transmitting a transmit signal from an external apparatus, the transmit signal including an RF signal that is amplitude modulated to include several notches; receiving, at an implantable pulse generator, an incident signal based on the transmit signal; and generating and delivering a train of mini-pulses to an electrode based on the incident signal, each mini-pulse having a voltage amplitude and a pulse width smaller than the pulse width TO, where delivery of the train of mini-pulses to the electrode effectively corresponds to a stimulation pulse having the pulse width TO and an amplitude that is a function of the pulse widths of the mini-pulses included in the train of mini-pulses.

An embodiment includes a stimulator that includes: an implantable pulse generator and having at least one electrode; and an external apparatus that includes a transmitter configured to generate and transmit an RF signal received by the implantable pulse generator, where the implantable pulse generator includes: an Rx antenna configured to receive a radio frequency (RF) signal; a rectifier coupled to the Rx antenna and configured to rectify the RF signal to generate an output voltage VDD; an energy storage capacitor coupled to the rectifier and configured to be charged by the output voltage VDD; a demodulator coupled to the Rx antenna, the rectifier, and a source, and configured to control an output of a train of mini-pulses, based on the RF signal, each mini-pulse having a voltage amplitude and a pulse width (on-portion) smaller than a pulse width TO; and stimulation circuitry coupled to receive the train of mini-pulses and deliver the train of mini-pulse to an electrode, the stimulation circuitry configured such that delivery of the train of mini-pulses to the electrode effectively corresponds to a stimulation pulse, having the pulse width TO and an amplitude that is a function of the pulse widths of the mini-pulses included in the train of mini-pulses.

In a further embodiment, the transmitter is configured to be programmed to control the configuration of the RF signal to thereby control the amplitude of the stimulation pulse.

In a further embodiment, the amplitude of the stimulation pulse is controlled by a number and duration of the notches in the RF signal, which duration controls the pulse widths of the mini-pulses in the train of mini-pulses.

### BRIEF DESCRIPTION OF THE DRAWINGS

FIG. 1 illustrates a stimulator having an implantable pulse generator (IPG) and an external apparatus, in accordance with an embodiment of the invention.

FIG. 2 illustrates a circuit architecture of an IPG in accordance with an embodiment of the invention.

FIG. 3 illustrates a circuit schematic of a demodulator of an IPG in accordance with an embodiment of the invention.

FIG. 4 illustrates a schematic of circuitry that simulates features of amplitude control and spectrum spreading enabled by a stimulator in accordance with an embodiment of the invention.

FIGS. 5A, 5B, 5C, and 5D, illustrate various stages of generation and delivery of two stimulation pulses based on different configurations of a train of mini-pulses, where each stimulation pulse has a 100 μsec pulse width and a different voltage amplitude, in accordance with an embodiment of the invention.

FIGS. 6A. 6B, 6C, 6D, 6E, and 6F illustrate various stages of generation and delivery of two stimulation pulses based on different configurations of a train of mini-pulses, where each stimulation pulse has a 100 usec pulse width and a same voltage amplitude, but the spectrum of the mini-pulses in the frequency domain are different, in accordance with an embodiment of the invention.

### DETAILED DESCRIPTION OF THE DRAWINGS

Turning now to the drawings, stimulators, including implantable pulse generators (IPGs) in accordance with various embodiments are illustrated. Many embodiments provide for stimulators with programmable amplitudes of produced signals.

### Circuit Implementations

### Stimulator

Stimulators with programmable amplitudes of produced signals in accordance with many embodiments of the invention are described. A stimulator can include an implantable pulse generator (IPG) and an external apparatus and the IPG can be configured to receive radio-frequency (RF) signals (which can be referred to as Tx signals and/or incident signals) from an external apparatus and to output stimulation pulses based on the RF signals. Stimulators in accordance with many embodiments can include an IPG that can be configured to harvest energy from RF signals. In many embodiments, a stimulator can produce a stimulation pulse with programmable amplitudes. In several embodiments, a stimulator can generate a train of mini-pulses with programmable spectrums in the frequency domain, where different frequency spectrums can be used to generate a same amplitude for a stimulation pulse.

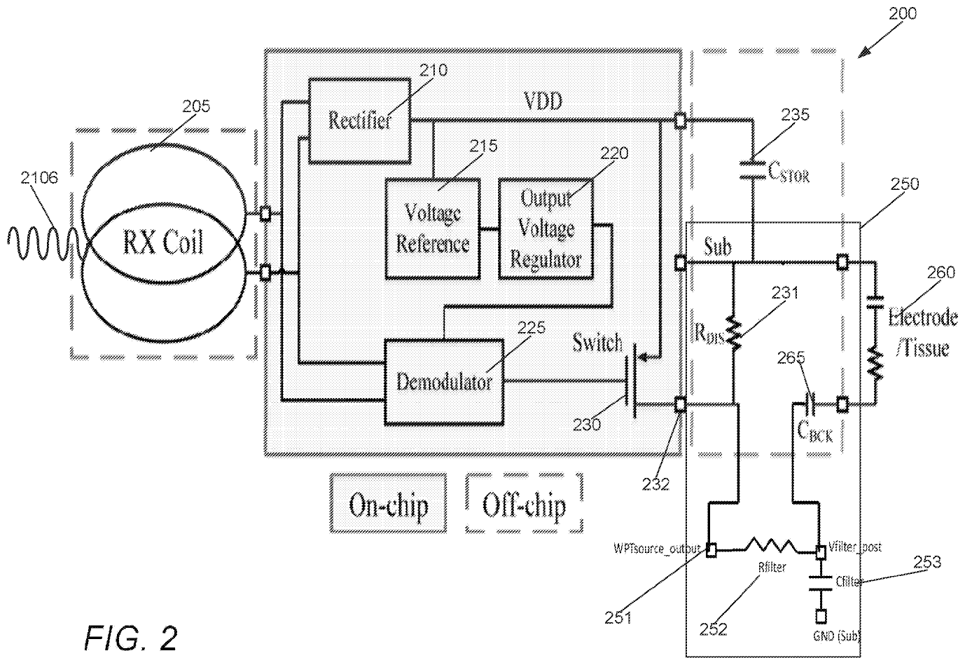

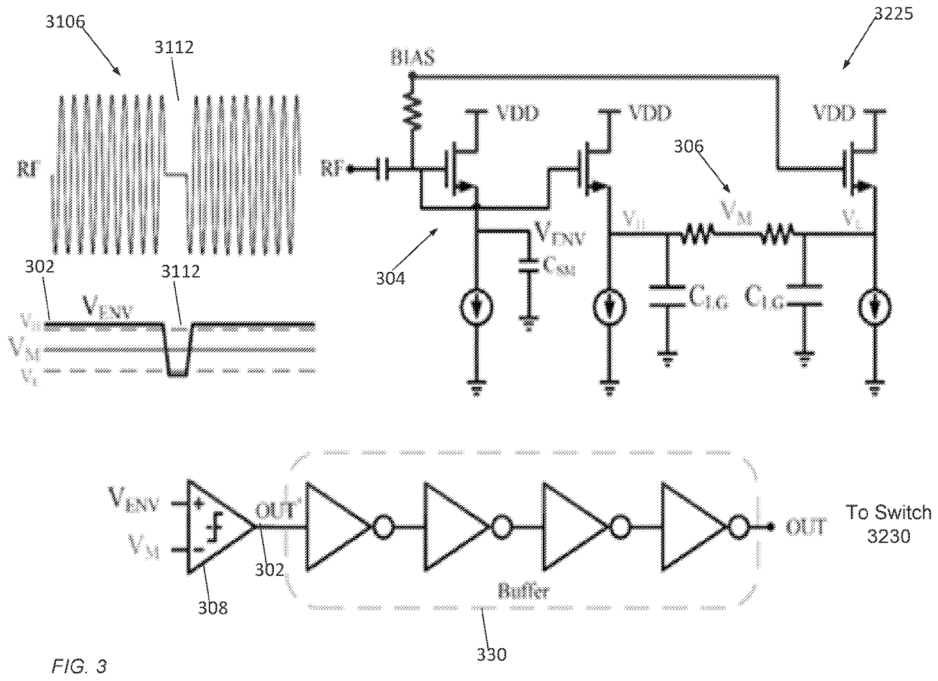

Systems and methods that use modulated RF signals to wirelessly power a device (e.g., an IPG of a stimulator) and to produce a stimulation pulse with programmable width (but not programmable amplitude and/or controllable frequency spectrums) are described in detail in U.S. Patent Publication 2022-0379124-A1, entitled “Wirelessly Powered Stimulator” filed Aug. 26, 2020 and published on Dec. 1, 2022, by Babakhani et al. which is herein incorporated by reference in its entirety, including in particular, FIG. 2A which illustrates a circuit architecture of an IPG, FIG. 2B which illustrates a schematic of a Tx coil, and FIG. 3 which illustrates a demodulator block that can be responsible for replicating the timing of a notch, and an output pulse produced and delivered to a load (e.g., electrode) of a wirelessly powered device (e.g., implant) as illustrated in FIG. 19.

However, stimulators can benefit from having a means to change an amplitude of a pulse. In particular, in many applications there can be a need to be able to program an amplitude of a produced signal independent of a pulse width delivered to a load (e.g., one or more electrodes). Accordingly, stimulators in accordance with many embodiments can provide programmability of an amplitude of a produced signal. In many embodiments, a programmability of an amplitude of a produced signal can be independent of a pulse width delivered to a load.

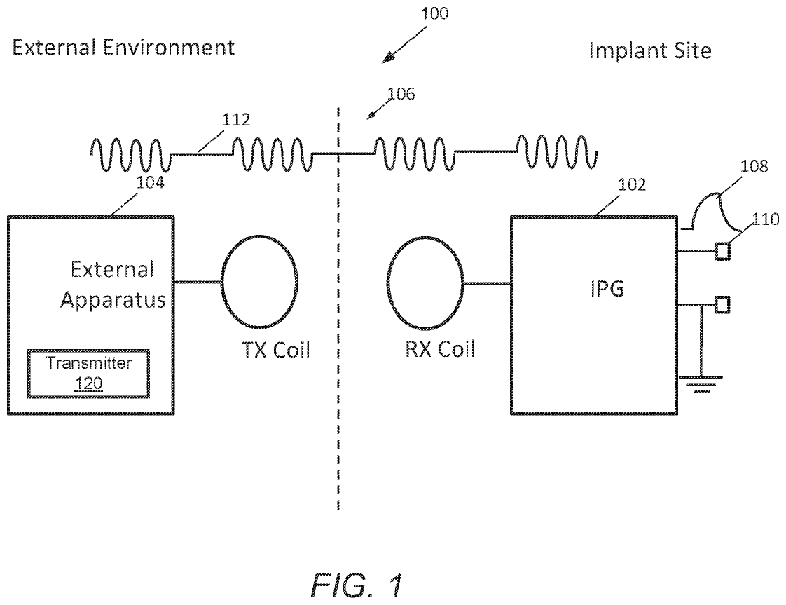

A stimulator 100 having an implantable pulse generator (IPG) 102 with programmable amplitudes of stimulation pulses and an external apparatus 104, in accordance with an embodiment of the invention is shown in FIG. 1. The IPG 102 can be configured to receive radio-frequency (RF) signals 106 (which can be referred to as Tx signals or incident signals) from an external apparatus 104 and to output stimulation pulses 108 to an electrode 110 based on the RF signals. The IPG 102 can also be configured to harvest energy from the RF signals 106. The stimulator 100 can produce a stimulation pulse with programmable amplitudes. In many embodiments, the programmable amplitudes can be independent of pulse widths delivered to a load.

As illustrated in FIG. 1 in accordance with several embodiments of the invention, a transmitter 120 of the external apparatus 104 can be configured to generate and transmit the RF signal 106 to the IPG 102. The transmitter 120 can also be configured to be programmed to control the configuration of the RF signal 106 to thereby control the amplitude of the stimulation pulse 108. In many embodiments, to this end, notches 112 can be intentionally included in the RF signal 106 to control the amplitude of the stimulation pulses 108. For example, a notch 112 may be included by reducing an amplitude of the RF signal 106 to a percentage of the peak amplitude that is used for purposes of energy harvesting by the IPG 102. The amplitude of the stimulation pulse 108 can be controlled by a number and/or duration of the notches 112 in the RF signal 106.

Although FIG. 1 illustrates a particular architecture of a stimulator that generates stimulation pulses with programmable amplitudes, any of a variety of architectures may be utilized for stimulators that generate stimulation pulses with programmable amplitudes as appropriate to the requirements of specific applications in accordance with embodiments.

### Implantable Pulse Generator (IPG)

Stimulators in accordance with many embodiments of the invention can include one or more IPGs that can provide amplitude control and/or spectrum spreading of stimulation pulses. A systematic architecture of an IPG that provides amplitude control and spectrum spreading in accordance with an embodiment of the invention is illustrated in FIG. 2. The IPG 200 can include an RX coil 205, a rectifier 210, a voltage reference 215, an output voltage regulator 220, a demodulator 225, a switch 230, an energy storage capacitor CSTOR 235, stimulation circuitry 250, and an electrode 260. The Rx coil 205 can be configured to receive RF signals 2106 (e.g., RF signal 106 of FIG. 1) that can be rectified by rectifier 210 to generate an output voltage (VDD). The output voltage VDD can charge the energy storage capacitor 235, CSTOR. The RF signals 2106 may also be received at a demodulator 225 and processed to control the on/off state of the switch 230. The on/off state of the switch 230 can control the on/off discharge of current from the energy storage capacitor 235 to provide mini-pulses to a stimulation circuitry 250. The stimulation circuitry 250 can be configured to provide amplitude control and/or spectrum spreading based on the mini-pulses.

In many embodiments, the output voltage VDD can be compared to a voltage reference 215 through the output voltage regulator 220. To this end, the output voltage regulator 220 can be configured to compare fractions of VDD with a constant voltage reference 215. In certain embodiments, when the output voltage VDD exceeds a first threshold value, e.g., 19/12 of the voltage reference 215, a discharge current path (not shown in FIG. 2) can be enabled to discharge the incident power. When the output voltage VDD is less than a second threshold value, e.g., 19/16 of the voltage reference 215, the output voltage regulator 220 can disable the demodulator 225. Circuitry within the output voltage regulator 220 can set the first and second threshold values of the voltage reference 215 to thereby regulate the amplitude of the mini-pulses within a specific range. In several embodiments, two or more threshold values can be specified to regulate an amplitude of mini-pulses with a specific range as appropriate to the requirements of specific applications in accordance with embodiments of the invention.

Although FIG. 2 illustrates a particular circuit architecture of an IPG configured to provide amplitude control and spectrum spreading of stimulation pulses, any of a variety of IPG circuit architectures may be utilized to provide amplitude control and spectrum spreading of stimulation pulses as appropriate to the requirements of specific applications in accordance with embodiments.

Stimulators in accordance with many embodiments can include a demodulator that can output a timing signal that replicates a timing of notches present in a Tx signal. A demodulator of an IPG that is configured to output a timing signal that replicates a timing of notices in a Tx signal in accordance with an embodiment of the invention is illustrated in FIG. 3. As illustrated in FIG. 3, a demodulator 3225 of an IPG (e.g., IPG 225 of FIG. 2 in accordance with an embodiment) can be configured to output a timing signal 302 that replicates the timing of notches 3112 present in a Tx signal 3106 (e.g., the notches 112 in TX signal 106 illustrated in FIG. 1 in accordance with an embodiment). The high end, low end, and transient envelope of the Tx signal 3106 (e.g., Tx signal 106 illustrated in FIG. 1) can be denoted as V_{H}, V_{L}, and V_{ENV}, respectively in the timing signal 302. The Tx signal 3106 can be input to circuitry 304 of a demodulator that includes a V_{ENV} detection branch, a V_{H} detection branch, and a V_{L} detection branch. The V_{ENV} detection branch may use a relatively small capacitor Cₛₘ to extract V_{ENV} from the Tx signal, while V_{H} and V_{L} can be extracted on larger capacitors with and without the AC input, respectively. The average V_{M}, e.g., the average of the high end V_{H} and the low end V_{L}, can be obtained through a resistive divider 306. The average V_{M} can be input to a comparator 308 and compared with V_{ENV} to reconstruct the timing of notches 3112 (e.g., notches 112 of FIG. 1) included in the TX signal 3106 (e.g., Tx signal 106 of FIG. 1). Capacitors Cₛₘ and C_{LG} can be selected to be e.g., 100 fF and e.g., 36 pF, respectively. As C_{SM} <<C_{LG}, the average V_{M} can be considered as constant so that the discharging and charging of C_{SM} can determine the delays from the starting point of a notch 3112 (e.g., notch 112 of FIG. 1) and the ending point of a notch, respectively. The timing signal at the output of the comparator 308 can then be sharpened by a following buffer 330 and then provided to the input of a switch 3230 (e.g., switch 230 of FIG. 2). Although FIG. 3 illustrates a particular circuit architecture of a demodulator that can output a timing signal that replicates a timing of notches in a Tx signal, any of a variety of circuit architectures of a demodulator that can output a timing signal that replicates a timing in a Tx signal may be utilized as appropriate to the requirements of specific applications in accordance with embodiments.

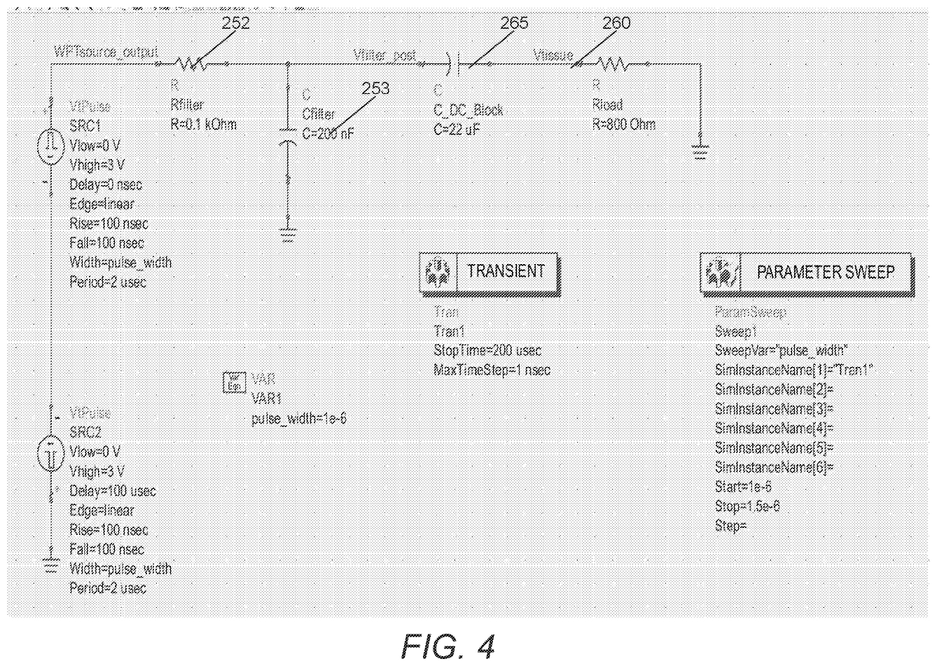

Returning to FIG. 2, and with additional reference to FIG. 4, stimulation circuitry 250 of the IPG 200 in accordance with many embodiments can include a low pass filter formed by Rfilter 252 and Cfilter 253, a DC-block capacitor 265, C_{BCK}, and a discharge resistor 231, RDIs. As described herein, the low pass filter can be configured to extract the average of the WPTsource_output 251 voltage. WPTsource_output voltage 251 can be the output 232 of the switch 230 and can be coupled to the electrode 260 through the stimulation circuitry 250. The DC-block capacitor 265, C_{BCK}, can provide charge-neutralization and can prevent any release of DC charge to the electrode 260. A discharge resistor 231, RDis, can null the accumulated charge on C_{BCK}.

Although FIGS. 2 and 4 illustrate a particular schematic of stimulation circuitry with programmable amplitudes of stimulation pulses, any of a variety of configurations can be utilized as appropriate to the requirements of specific applications in accordance with embodiments.

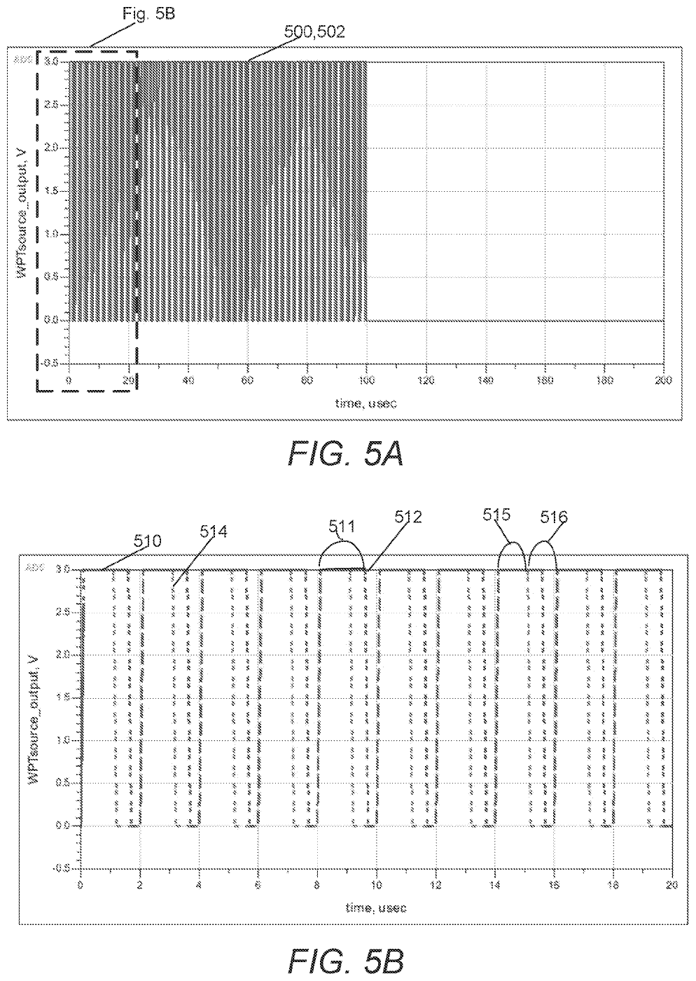

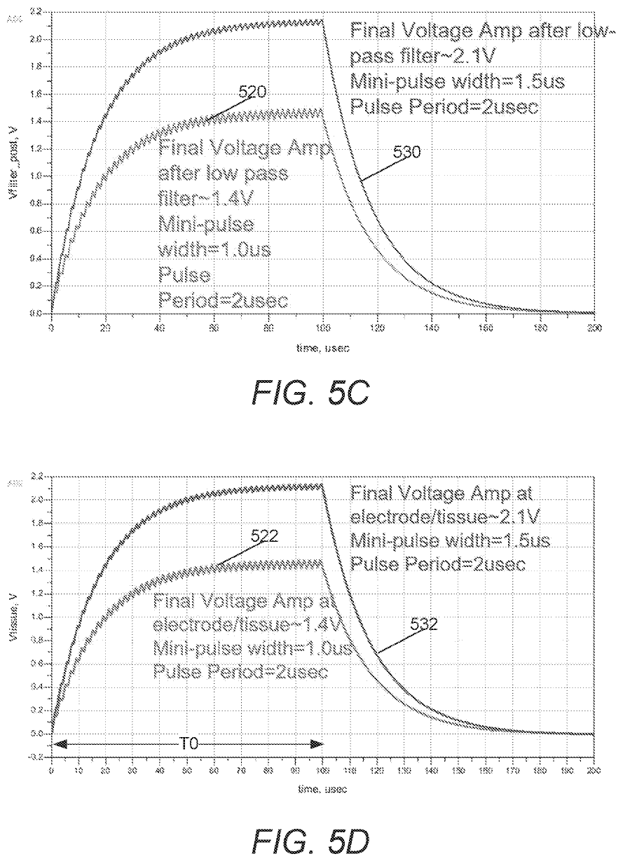

FIGS. 5A-5D illustrate various graphs with respect to generating a stimulation pulse using a train of mini-pulses having a programmable amplitude in accordance with an embodiment of the invention. With reference to FIGS. 5A-5D, stimulators in accordance with several embodiments can include stimulation circuitry (e.g., stimulation circuitry 250 illustrated in FIG. 2) that can provide stimulation pulses 522, 532 of different amplitudes by producing a corresponding train of mini-pulses (illustrated as 500, 502 in FIG. 5A and illustrated in a zoomed in graph of the train of mini-pulses 510, 514 illustrated in FIG. 5B) of different configurations. For example, referring to FIGS. 5A and 5B, a train 500 of 50 mini-pulses 510, each having an on-portion 511 and an off-portion 512, which are delivered to an output node (e.g., WPTsource_output node of the stimulation circuitry 250 of FIG. 2) can produce: 1) a pulse (e.g., pulse 530 in FIG. 5C) at a node (e.g., Vfilter_post node of the stimulation circuitry in FIGS. 2) and 2) a stimulation pulse (e.g., stimulation pulse 532 in FIG. 5D) at an electrode (e.g., electrode 260 of FIG. 2).

With continued reference to FIGS. 5A and 5B, a train 502 of 50 mini-pulses 514, each having an on-portion 515 and an off-portion 516, which can be delivered to an output node (e.g., WPTsource_output node 251 of stimulation circuitry 250 of FIG. 2) can produce: 1) a pulse 520 (as shown in FIG. 5C) at a node (e.g., Vfilter_post node of stimulation circuitry 250 of FIGS. 2) and 2) a stimulation pulse 522 (as shown in FIG. 5D) at an electrode (e.g., electrode 260 of FIG. 2). Note that the differences in the durations of the on-portions and off-portions of the respective trains 500, 502 of mini-pulses results in stimulation pulses 522, 532 of different amplitudes. Thus, stimulators in accordance with many embodiments can be configured to control an amplitude of stimulation pulses (e.g., stimulation pule 522, 532) based on a configuration of a train of mini-pulses (e.g., train 500, 502 of mini-pulses illustrated in FIG. 5A).

With reference to FIG. 5D, stimulators in accordance with many embodiments can produce a stimulation pulse 522, 532 having an effective voltage and/or current output pulse (e.g., Pulse-0) with a duration of TO. Stimulators in accordance with many embodiments can be used in many different medical applications, and TO in many medical applications can range from e.g., 10 usec to 100 msec, among various other ranges as appropriate to the requirements of specific applications in accordance with embodiments of the invention. Stimulators in accordance with many embodiments can produce a stimulation pulse 522, 532 with duration TO by producing a train 500, 502 of smaller pulses 510, 514 (e.g., mini-pulses) such that their individual duration (e.g., a summation of their on portion and off portion) can be significantly smaller than TO. An individual duration of a mini-pulse 510, 514 can be smaller than TO by at least a factor of 10 or larger. Stimulators in accordance with many embodiments of the invention can use N mini-pulses 510, 514 with durations of T1,T2, . . . , TN. In a practical example, 0.1 μsec<T1, . . . TN<1 msec. T1, TN may need to have the following condition:

$$T 1 + T 2 + … + TN < T 0. \qquad ( condition 1 )$$

Stimulators in accordance with many embodiments of the invention can pass a train of mini-pulses, illustrated as train 500 and 502 in FIG. 5A, with a zoomed in illustration of the train 510, 514 in FIG. 5B (Pulse-1, . . . , Pulse-N) with respective durations of T1, . . . ,TN through a low pass filter, such as a low pass filter illustrated in FIGS. 2 and 4 in accordance with an embodiment, to produce a single voltage pulse, e.g., a stimulation pulse 522, 532 of FIG. 5D, with a duration of TO such that its amplitude is almost proportional to (T1+ . . . +TN)/TO. With these techniques, a single stimulation pulse (e.g., stimulation pulse 522, 532 of FIG. 5D) can be generated with a peak amplitude of VO such that VO is proportional to (T1+ . . . +TN)/TO.

Stimulators in accordance with many embodiments can control an amplitude of a pulse (e.g., Pulse-0) by controlling a ratio (T1+ . . . +TN)/TO. Stimulators in accordance with many embodiments can control a spectrum of a pulse (e.g., Pulse-0) in the frequency domain while keeping its voltage VO constant with choosing different values for (T1, . . . , TN) such that (T1+ . . . +TN)/TO remains constant.

Stimulators in accordance with several embodiments of the invention may produce and deliver a particular usec signal (e.g., 100 usec signal) to a load. In many embodiments, instead of turning off a wireless RF signal to produce a particular usec pulse duration (e.g., 100 usec pulse may be used for the particular example illustrated in this paragraph) at an output of a wirelessly powered device, many embodiments can produce a train (e.g., train 500, 502 of FIG. 5A) of mini-pulses (e.g., 510, 514 of FIG. 5B) within the particular time duration (e.g., 50 pulses within 100 usec). If a duration of each mini-pulse (e.g., 510, 514 of FIG. 5B) is a particular time (e.g., 2 μsec), and starting times of two consequent mini-pulses are separated by a particular time (e.g., 2 μsec), then these pulses (e.g., 50 pulses) can produce a particular stimulation pulse (e.g., 522, 532 of FIG. 5D, providing e.g., 100 μsec pulse). If a width of each mini-pulse (e.g., 510, 514 of FIG. 5B) is reduced to a shorter particular time (e.g., 1 μsec) which can provide a particular duty cycle. (e.g., 50% duty-cycle) and the starting times of two consequence mini-pulses separated by a particular time (e.g., 2 μsec) is kept the same, then a stimulator can in essence produce a number or train of mini-pulses (e.g., 50 mini-pulses) each with duration of a particular time (e.g., 1 μsec within 100 μsec time). This can result in total on time of, e.g., 50×1 sec=50 μsec and total off time of 50 sec within 100 μsec.

Considering FIGS. 5A-5D further, simulation results show that changing a pulse width (on-portion) of each mini-pulse 510, 514 in a train 500, 502 of 50 mini-pulses from 1 μsec (on-portion 515 of mini-pulses 514) to 1.5 μsec (on-portion 511 of mini-pulse 510) (e.g., a 50% increase in pulse width) can increase an amplitude of a stimulation pulse by 50% from ˜1.4V (stimulation pulse 522) to ˜2.1 (stimulation pulse 532).

In many embodiments, a stimulator may be configured to produce a pulse train of several e.g., 50 mini-pulses within a time period e.g., 100 μsec (e.g., mini-pulses), where the pulse width (on-portion) of each mini-pulse can be a certain duration e.g., 2 μsec, and a time difference between a starting time of two consequent mini-pulses is a certain duration e.g., 2 μsec. Configured as such, a pulse train of e.g., 50 mini-pulses can produce an e.g., 100 μsec pulse. In accordance with an embodiment, and with reference to FIGS. 5A-5D, if a pulse width (on-portion) of each mini-pulse is reduced from 2 μsec to 1 μsec (50% duty-cycle) and the time difference between the starting time of two consequence mini-pulses is kept at 2 μsec, a stimulator can produce a pulse train 502 of 50 mini-pulses 514, each with a pulse width (on-portion) of 1 μsec within 100 μsec time. Similarly, if a pulse width (on-portion) of each mini-pulse is reduced from 2 μsec to 1.5 μsec (75% duty-cycle) and the time difference between the starting time of two consequence mini-pulses is kept at 2 μsec, a stimulator can produce a pulse train 500 of 50 mini-pulses 510, each with a pulse width (on-portion) of 1.5 μsec within 100 μsec time. As illustrated in FIG. 5D, the pulse train configuration of mini-pulse width (on-portion)=1.0 μs, pulse period=2 μsec produces a stimulation pulse 522 with a final voltage amp at electrode/tissue ˜1.4V, while the pulse train configuration mini-pulse width (on-portion) =1.5 μs, pulse period=2 μsec produces a stimulation pulse 532 with a final voltage amp at electrode/tissue ˜2.1V.

In many embodiments, if a pulse train 500, 502 of mini-pulses 510, 514 is generated and passed through a low-pass filter (e.g. RC, LC, and/or other), then a particular stimulation pulse having a pulse duration and an amplitude based on the total on time (the summation of the on-portions 511, 515 of the mini-pulses 510, 514) compared to the total off time (the summation of the off-portions 512, 516 of the mini-pulses 510, 514) within the particular time e.g., 100 μsec.

Stimulators in accordance with many embodiments may be configured to generate different trains of mini-pulses over several windows, where each train includes differently configured mini-pulses. For example, during a first 100 μsec window, a first train of 50 mini-pulses each with a pulse width (on-portion) of 1 μsec (50% duty cycle) can be used, while during a second 100 μsec window a second train of 25 mini-pulses each with a pulse width (on-portion) of 2 μsec (50% duty cycle) can be used. The configurations of the trains (i.e., the number of pulses within a window, the pulse width, duty cycle, among other settings) may alternate back and forth between the two (or more) configurations. In certain embodiments, additional pulse train configurations may be used. In several embodiments, a train may be randomly configured (have random number of mini-pulses, random pulse width (on-portion), random duty cycle, among others).

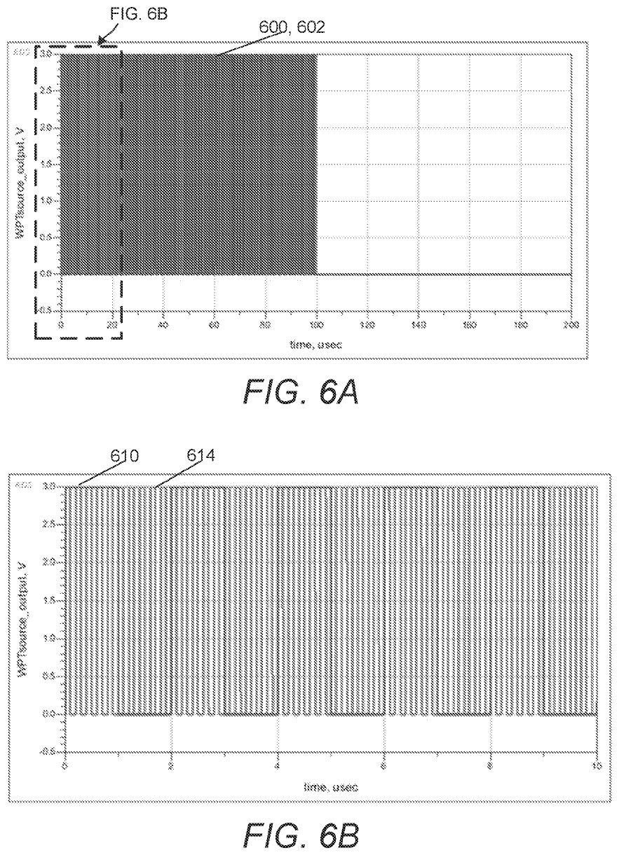

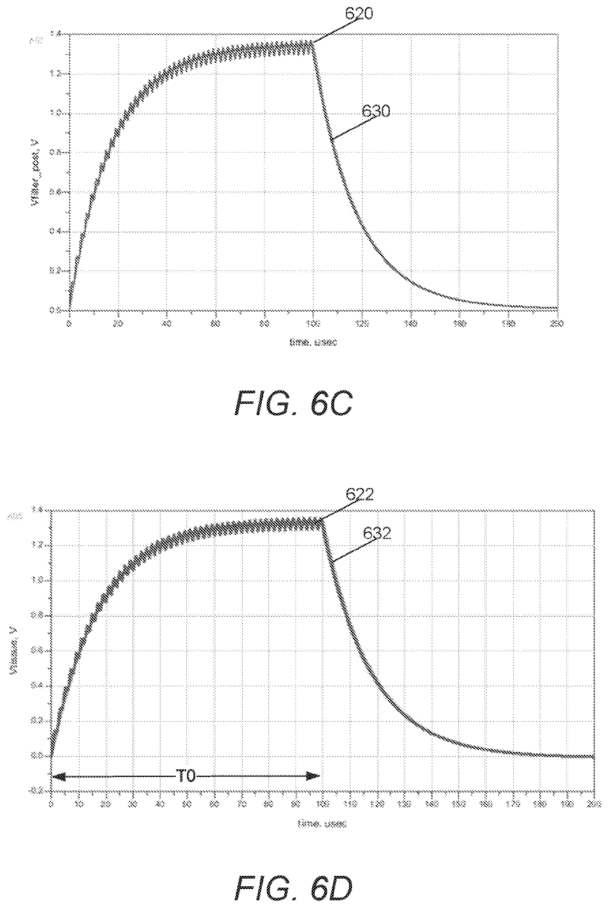

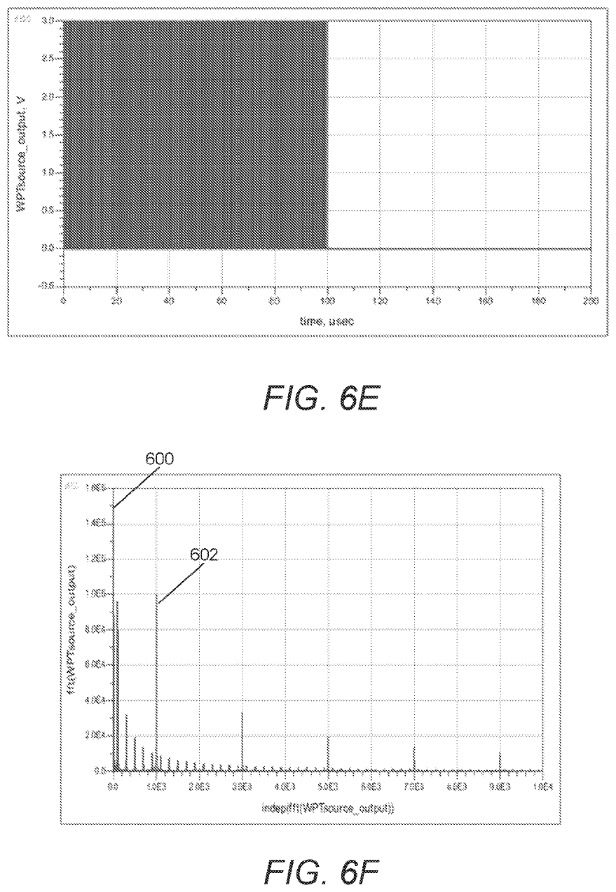

FIG. 6A-6F illustrate various graphs with respect to generating a stimulation pulse using a train of mini-pulses having a controllable spectrum in accordance with an embodiment of the invention. With reference to FIGS. 6A-6F, a first train 600 of 50 mini-pulses 610 having a duration (or period) of 2 μsec, a pulse width (on-portion) of 1 μsec produce: 1) a pulse 620 at a node of a stimulation circuitry (e.g., Vfilter_post node of a stimulation circuitry 250 of FIGS. 2) and 2) a stimulation pulse 620 at an electrode (e.g., electrode 260 of FIG. 2) a stimulation pulse 622 having a duration of 100 μsec and an amplitude of ˜1.3V. A second train 602 mini-pulses 614 having a duration (or period) of 0.2 μsec, a pulse width (on-portion) of 0.1 μsec produce: 1) a pulse 630 at a node of a stimulation circuitry (e.g., Vfilter_post node of stimulation circuitry 250 of FIGS. 2) and 2) a stimulation pulse 632 at an electrode (e.g., electrode 260 of FIG. 2), a stimulation pulse 622 having a duration of 100 μsec and an amplitude of ˜1.3V.

With reference to FIG. 6F, a Fourier transform of the respective trains 600 (blue), 602 (red) at an output note of a stimulation circuitry (e.g., WPTsource_output node of stimulation circuitry 250 of FIG. 2) reveals that the spectrum of a train of mini-pulses in the frequency domain changes based on the configuration of the train. As illustrated in FIG. 6F, the spectrum of the train 600 (blue) has less spurs than the spectrum of the train 602 (red). Thus, in accordance with embodiments, a stimulator may be configured (e.g. programmed) to generate a stimulation pulse using a train of mini-pulses having a desirable spectrum in the RF domain (e.g., signal 106). For example, a desirable spectrum in the RF domain can be an RF signal with a least amount of spurs and/or one below emission levels allowed in various different standards. Accordingly, stimulators in accordance with many embodiments can provide spectrum control, where a spectrum of an RF signal can be controlled and can change to a desirable spectrum in the RF domain, and a stimulator can be configured to deliver the same stimulation pulse with a duration TO to a tissue for the changing spectrums of the RF signals in the RF domain.

Although specific implementations for stimulators with programmable amplitude levels are discussed above with respect to FIGS. 1-6F, any of a variety of implementations utilizing the above discussed techniques can be utilized for stimulators with programmable amplitudes in accordance with embodiments. While the above description contains many specific embodiments, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. An implantable pulse generator 200 comprising:
an Rx antenna 205 configured to receive a radio frequency (RF) signal 106;
a rectifier 210 coupled to the Rx antenna and configured to rectify the RF signal to generate an output voltage VDD;
an energy storage capacitor 235 coupled to the rectifier and configured to be charged by the output voltage VDD;
a demodulator 225 coupled to the Rx antenna, the rectifier, and a source, and configured to control an output of a train 400 of mini-pulses 410, 414 based on the RF signal, each mini-pulse having a voltage amplitude and a pulse width (on-portion) smaller than a pulse width TO; and
stimulation circuitry 250 coupled to receive the train 400 of mini-pulses and deliver the train of mini-pulse to an electrode 260, the stimulation circuitry configured such that delivery of the train of mini-pulses to the electrode effectively corresponds to a stimulation pulse 422, 432 having the pulse width TO and an amplitude that is a function of the pulse widths of the mini-pulses included in the train of mini-pulses.

2. The implantable pulse generator of claim 1, wherein the RF signal 106 is amplitude modulated to include a plurality of notches 112, and the demodulator 225 is configured to detect the plurality of notches and, for each notch, to enable the output of a mini-pulse in the train of mini-pulses.

3. The implantable pulse generator of claim 1, wherein the demodulator 225 is configured to control a release of energy stored in an energy storage capacitor 235 to output the train of mini-pulses.

4. The implantable pulse generator of claim 1, wherein the demodulator 225 is configured to control the coupling of the output voltage VDD to output the train of mini-pulses.

5. The implantable pulse generator of claim 1, wherein the stimulation circuitry 250 comprises a low pass filter.

6. The implantable pulse generator of claim 5, wherein the low pass filter comprises a resistor 252 and a capacitor 253 that are added.

7. The implantable pulse generator of claim 5, wherein the stimulation circuitry comprises a DC-block capacitor 265 coupled to the output of the low pass filter, wherein the DC-block capacitor prevents release of DC charge.

8. The implantable pulse generator of claim 1, wherein:
each mini-pulse comprises an on portion and an off portion, the on portion corresponding to the pulse width of the mini-pulse; and
the pulse width TO of the stimulation pulse is greater than a summation of the on portions of the mini-pulses in the train of mini-pulses.

9. The implantable pulse generator of claim 1, wherein:
each mini-pulse comprises an on portion and an off portion, the on portion corresponding to the pulse width of the mini-pulse; and
the pulse width TO of the stimulation pulse is substantially equal to a summation of the on portion and the off portion of each mini-pulse in the train of mini-pulses.

10. The implantable pulse generator of claim 1, wherein:
each mini-pulse comprises an on portion and an off portion, the on portion corresponding to the pulse width of the mini-pulse; and
the amplitude of the stimulation pulse is proportional to a summation of the pulse widths of the mini-pulses in the train of mini-pulses.

11. The implantable pulse generator of claim 1, wherein the voltage amplitudes of the mini-pulses in the train of mini-pulses are constant.

12. The implantable pulse generator of claim 1, wherein the pulse widths of the mini-pulses in the train of mini-pulses are constant.

13. The implantable pulse generator of claim 1, wherein the pulse widths of the mini-pulses in the train of mini-pulses vary.

14. The implantable pulse generator of claim 1, wherein the pulse widths of the mini-pulses in the train of mini-pulses alternate between a first pulse width and a second pulse width.

15. The implantable pulse generator of claim 1, wherein a first subset of the mini-pulses in the train of mini-pulses have a first pulse width and a second subset of the mini-pulses in the train of mini-pulses have a second pulse width different from the first pulse width.

16. The implantable pulse generator of claim 1, wherein the train of mini-pulses is configurable to provide different spectrums in the frequency domain, wherein a spectrum of a train of mini-pulses in the frequency domain changes based on a configuration of the train of mini-pulses.

17. The implantable pulse generator of claim 1, wherein a spectrum of the RF signal 106 changes in the frequency domain, wherein the stimulation circuitry generates a same stimulation pulse having the pulse width TO.

18. The implantable pulse generator of claim 1, wherein the stimulation circuitry 250 is coupled to receive different trains of mini-pulses in the frequency domain and generate a same stimulation pulse having the pulse width TO and an amplitude that is a function of the pulse widths of the mini-pulses included in the different trains of mini-pulses.

19. A method of delivering a stimulation pulse having a pulse width TO and an amplitude, the method comprising:
transmitting a transmit signal 112 from an external apparatus 104, the transmit signal comprising an RF signal 112 that is amplitude modulated to include a plurality of notches;
receiving, at an implantable pulse generator 102, an incident signal 106 based on the transmit signal 112; and
generating and delivering a train of mini-pulses 108 to an electrode 110 based on the incident signal, each mini-pulse having a voltage amplitude and a pulse width smaller than the pulse width TO,
wherein delivery of the train of mini-pulses to the electrode effectively corresponds to a stimulation pulse having the pulse width TO and an amplitude that is a function of the pulse widths of the mini-pulses included in the train of mini-pulses.

20. A stimulator comprising:
an implantable pulse generator 102 configured in accordance with claim 1 and having at least one electrode 110; and
an external apparatus 104 comprising a transmitter 120 configured to generate and transmit the RF signal 112 received by the implantable pulse generator as recited in claim 1.

21. The stimulator of claim 20, wherein the transmitter 120 is configured to be programmed to control the configuration of the RF signal 106 to thereby control the amplitude of the stimulation pulse 108.

22. The implantable pulse generator of claim 21, wherein the amplitude of the stimulation pulse 108 is controlled by a number and duration of the notches 112 in the RF signal 106, which duration controls the pulse widths of the mini-pulses in the train of mini-pulses.
